(19) United States
(12) Patent Application Publication
(10) Pub. No.: US 2014/0026260 A1
(43) Pub. Date: Jan. 23, 2014

(54) PLANT AND FUNGI MICROPROPAGATION METHODS AND PRODUCTS MADE THEREBY

(71) Applicant: Pamela J. Weathers, Stow, MA (US)

(72) Inventor: Pamela J. Weathers, Stow, MA (US)

(21) Appl. No.: 13/985,114

(22) PCT Filed: Feb. 17, 2012
(86) PCT No.: PCT/US12/25630

§ 371 Date: Oct. 8, 2013

Related U.S. Application Data
(60) Provisional application No. 61/443,875, filed on Feb. 17, 2011.

(51) Int. Cl. C12M 1/12; A01G 1/00; C12P 1/02

(52) U.S. Cl. CPC C12M 25/14; C12P 1/02; A01G 1/001
USPC 800/298; 435/180; 435/171; 435/144; 47/58.1 R

(57) ABSTRACT

Methods for micropropagation of plant or fungal material within a plant bioreactor by attachment of the plant or fungal material to a positively-charged polymer-coated support structure and placement of the support structure within a growth chamber of the bioreactor are described.

### BACKGROUND

Micropropagation is a widely applied technique for rapid regeneration of whole plants in large numbers using plant tissues ranging from single cells to shoot segments. Micropropagation is a very labor intensive technology that is, however, critically important for large, rapid production of elite plant genotypes. As a result of the high costs of this labor intensive activity, the micropropagation industry, originally developed decades ago in the U.S., has had to move much of its production offshore.

Although development of automated micropropagation systems seems like a reasonable goal, the technical hurdles are at best challenging for a number of reasons: 1) Micropropagation plantlets may have large value in bulk, but per plant they are worth very little—a few cents—so automation must be very low cost; 2) Plants have diverse growth morphologies making automation difficult—e.g. a strap-like leafy plant looks very different from a lacey one, so machine vision or robotics is not practical, nor cost effective; 3) Plants go through many developmental stages before they can be moved from lab to field. These stages begin with an in vitro inoculation of cells or tissues, then alteration of medium for growth into shoots with roots, and then by alteration of ambient gases and humidity acclimatization to toughen them up to withstand field conditions; 4) Even with inexpensive automation, plants have to be harvested, so if they are produced in any form, but in a tangled mass (e.g. rooted and thus tangled) they will require much labor to disentangle for planting resulting in increased labor costs; and 5) Many bioreactors have been developed with the intent of bulk production of plants, but none has facilitated either a single step culture ready for the field or facilitated easy harvest of the large amount of biomass produced after significant multiplication has occurred.

Cells of many species of plants can undergo spontaneous asexual development into new young plants by formation of an embryo clone from an undifferentiated cell (embryogenesis). Indeed, any plant cell has the potential to do this. Likewise, bits (explants) of plant tissue (e.g. plant leaves, stems, roots or reproductive material (male or female tissues or cells)) can also be manipulated by hormone changes to give a fully developed plant. This totipotency feature of plants is the basis of the international micropropagation industry. Using a mist bioreactor, previous studies showed it is possible to do a one-step acclimatization by manipulating culture conditions to convert inoculated shoots into fully rooted and acclimatized plants that withstood field conditions (see, Correll, M. J. et al. (2001), “Controlling hyperhydration of carnations (Dianthus caryaphyllus L.) grown in a mist reactor,” Biotechnology & Bioengineering 71:307-314; Correll, M. J. et al. (2001), “Effects of light CO2, and humidity on carnation growth, hyperhydration, and cuticular wax development in a mist reactor,” In Vitro Cellular and Developmental Biology-Plant, 37:405-413; Correll, M. J. et al. (2001), “One-step acclimatization of plantlets using a mist reactor,” Biotechnology and Bioengineering, 73:253-258; incorporated by reference herein). The improved development of a nutrient mist reactor to a reliable, scalable system, described in Liu, C. Z. et al. (2009), “Production of mouse interleukin-12 is greater in tobacco hairy roots grown in a mist reactor than in an airlift Reactor,” Biotechnol Bioengin, 102:1074-1086; and Weathers, P. J. et al. (2010), “Bench to batch: Advances in plant cell culture for producing useful products,” Applied Microbiology Biotechnology, 85:1339-1351 (both incorporated by reference herein), has made possible technologies that enable such a mist reactor to be useful in many sectors of the plant science industry.

Similar challenges apply to the micropropagation of fungi. Nearly all fungi have a filamentous stage, but for production of useful products, most fungi in this form are grown best not in liquid, where mass and gas transfer are extremely challenging, but rather in a solid-state fermentor (SSF). A SSF involves trickling nutrient medium over a usually horizontal bed of fungi to facilitate growth and product yield. Rapidly growing fungi have high respiration rates and require large amounts of oxygen while also producing heat, so use of a mist reactor as described above would significantly enhance fungal growth, while offering a completely new design of a SSF. Product would be excreted and then drip off the bed of fungi for collection and then product purification. Citric acid production, a low value, high quantity commercial product, is an example whereby SSF is used commercially. However, there are many small molecules, such as antibiotics, that are produced by fungi that have higher value and where an improved SSF technology using sterile reactors and axenic cultures are useful because these molecules impact human health.

Fungi grow best in a high humidity atmosphere where nutrients are readily available and temperature and gas content can also be altered. The mist bioreactor described above offers such an environment. However, to grow in what is essentially a liquid-laden gas phase, the fungi must be suspended or they will amass into balls of filaments that will trap water and then suffer from waterlogging and poor gas exchange. Mass and gas transfer would also be far from ideal.

It would therefore be useful to develop a low-cost, high-yield method for micropropagation of plant cells or tissues resulting in fully developed plantlets acclimatized and ready for planting.

It would also be useful to develop a safe, high-yield method for organizing and facilitating fungi for solid-phase culture, resulting in a high-quality product.

### SUMMARY OF INVENTION

The methods of these teachings relates to either cells or tissue bits (e.g. explants). Cells or tissue bits are inoculated onto a treated strip of material to which they then bind, and by changing media and culture conditions, they develop into plants with fully-formed rooted shoots, acclimated and ready for the field or, in another embodiment, into filamentous fungi ready for cultivation.

In one embodiment of the methods of these teachings, plants are micropropagated by coating at least part of a support structure with a positively-charged polymer, attaching plant materials to the polymer-coated support structure, placing one or more of the support structures inside of a growth chamber of a plant bioreactor, allowing the plant materials to develop into plantlets, optionally acclimating the plantlets to an environment outside of the plant bioreactor, and then harvesting the plantlets.

In an alternative embodiment of the methods of these teachings, fungi are cultivated by coating at least part of a support structure with a positively-charged polymer, attaching fungal materials to the polymer-coated support structure, placing the support structure inside of a growth chamber of a plant bioreactor, flowing a culture medium over the attached fungal materials, optionally allowing the fungal materials to develop into a filamentous form, and collecting an excreted product of the fungi.

### BRIEF DESCRIPTION OF THE DRAWINGS

FIGS. 1a-h illustrate variations of a plant bioreactor;

FIG. 2 is a conceptual illustration of hanging plants growing off strips to which they were attached as inoculum in a mist reactor growth chamber;

FIGS. 3a-c are a conceptual illustration of positively-charged polymer-coated support structure;

FIG. 4 is a conceptual illustration of plantlets on a positively-charged polymer-coated support structure in a rectangular culture vessel, e.g. a rocker box;

FIG. 5a shows a chart of viability of carrot cells after spraying;

FIG. 5b is a photographic illustration of embryos in reactor after spray inoculation;

FIG. 5c is a graph of attachment kinetics of cells to poly-L-lysine (PL) coated nylon or polypropylene mesh or sheeting;

FIG. 5d is a graph showing the effects of nutrient flow rate and misting cycle on embryo development;

FIG. 6 is a graph of incubation times versus leaf tissue binding to both PL coated nylon and polypropylene materials;

FIG. 7 is a chart comparing the binding of in vitro leafy tissue with greenhouse leaves;

FIG. 8 is a photographic illustration of in vitro cultured Artemisia annua leaf;

FIG. 9 is a photographic illustration of plantlets that formed from carrot cells inoculated onto a PL coated polypropylene strip and hung and then grown inside a mist bioreactor;

FIG. 10 is a graph showing adhesion of A. annua leaf explants to nylon 70 mesh (N70); and

FIGS. 11a-b are photographic illustrations of the lateral and top views of a conceptual trellis structure that could be coated with positively-charged polymer for fungal attachment and subsequent growth.

### DETAILED DESCRIPTION

### Micropropagation of Plant Cells

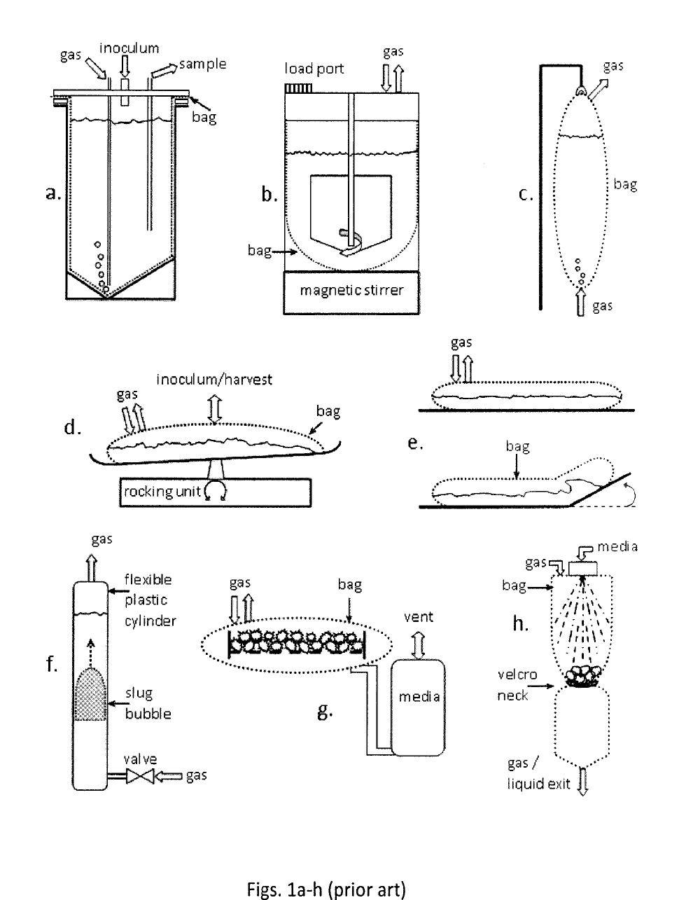

In one embodiment of the invention described herein is the successful demonstration of plant cell to embryo development within a plant bioreactor (FIGS. 1a-g) and more particularly within a nutrient mist plant bioreactor (FIG. 1h). A more detailed example of a mist bioreactor as described below can be found in U.S. Pat. No. 8,114,664, incorporated by reference herein.

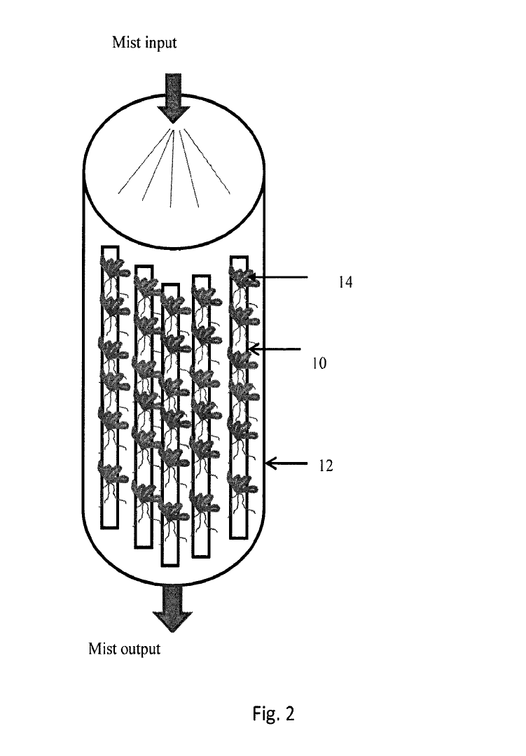

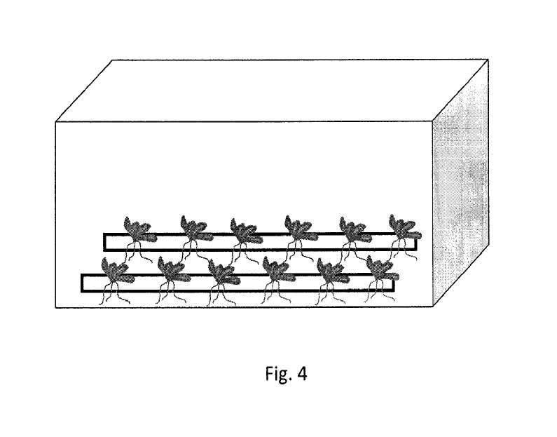

In one embodiment of these teachings, cells are inoculated by misting them into the bioreactor with little loss of viability. Once in the reactor, they form viable embryos. Towards scaling the technology up, the cells are attached to a support structure, such as strips of a poly-L-lysine (PL) coated synthetic films, sheets, or screens (e.g. polypropylene, nylon, polycarbonate, polystyrene etc.) so that the strips can be hung in multiples within a plant bioreactor growth chamber (FIG. 2) where they can be fed any gas and liquid nutrient medium and developed into fully acclimatized plants for field transplant. Other positively-charged polymers, such as poly-Larginine, poly-L-ornithine or poly-L-histidine, could also be used for coating the support structure. Likewise, the adhered cells on a strip could be placed inside a more horizontal bioreactor currently in use, such as rectangular rocking boxes that are slowly rocked to swish shallow medium back and forth (FIG. 4), temporary immersion reactors (FIG. 1g) or even incorporated into a wave reactor (FIGS. 1d-e).

Because many plants are preferably propagated in the industry using shoots (leafy tissues), the teachings herein show that the same procedure will also work with leafy tissue bits adhered to the polymer-coated strips. Again by changing the conditions within the reactor growth chamber, fully acclimated plants can be produced for the field.

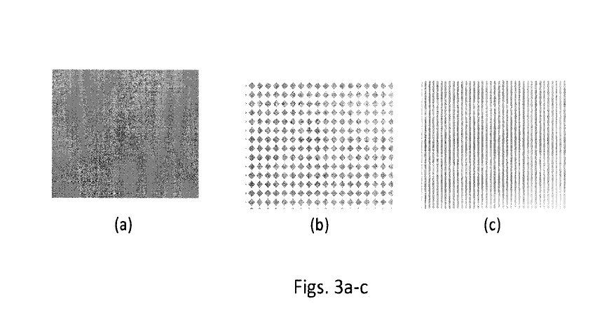

In the methods described herein, strips 10 of flexible films, screens or sheets can be used. This material is first sterilized by autoclaving and then coated with PL by immersing the material in a filter-sterilized 0.1% (w/v in water) solution of PL. The material is air-dried in a sterile hood prior to attachment of the cells or tissues. Attachment is effected by immersing the material in a solution of cells or tissues and waiting (e.g. 0.5-4 hrs) for attachment to occur. The strip 10 can be coated with PL in its entirety as depicted in FIG. 3a, or applied as organized segments or patterns, such as blocks, triangles, ovals, dots, stripes, zig-zags etc., examples of which are shown in FIGS. 3b-c, using a segmented applicator. By segmenting the PL, it is possible to spatially isolate inoculum material to enhance quality of plant growth and minimize harvesting crossover of tissues (e.g. overlapping leaves). For each species of plant, there is an optimum spacing as is known already within the industry, and such spacing enhances plantlet quality because there is improved gas circulation around the plantlets and less direct competition for soluble nutrients (e.g. nitrate, phosphate etc). Thus, a user would want to establish what the optimum spacing would be per species, or more likely, per group of species that have similarly shaped leaves. If the strip 10 is hung in a vertical mist reactor, such as in FIG. 2, the PL coating can be on both sides and plant materials can be attached and grown on both sides, thereby maximizing the number of plants grown within a vertical growth chamber 12.

Any of these applications of PL can be used on one side of the strip 10 and enable use of the invention within many versions of nonvertical bioreactors. Most reactors are horizontally configured for micropropagation and include versions like rocker boxes (FIG. 4) or wave reactors (FIGS. 1d-e), so then the organization of growing plantlets 14 could occur as depicted in FIG. 4, and plantlets 14 could readily be organized into optimally spaced growing patterns ready for harvest. A drawback to this form of an embodiment is that only one side of the strip 10 can be cultured as opposed to both sides when the cultures are vertically hung; this results in 50% fewer plants per unit space. Thus, a vertical culture space would increase production by at least 50%.

There are two modes whereby cells can be attached onto the strips 10: 1) by immersion in a solution of cells, such as when the strips, especially mesh strips, are sterilely dipped into a solution of cells, or when a solution of cells is sprayed onto strips already hanging in the bioreactor or; 2) by sterilely pipetting a solution of cells onto the strips.

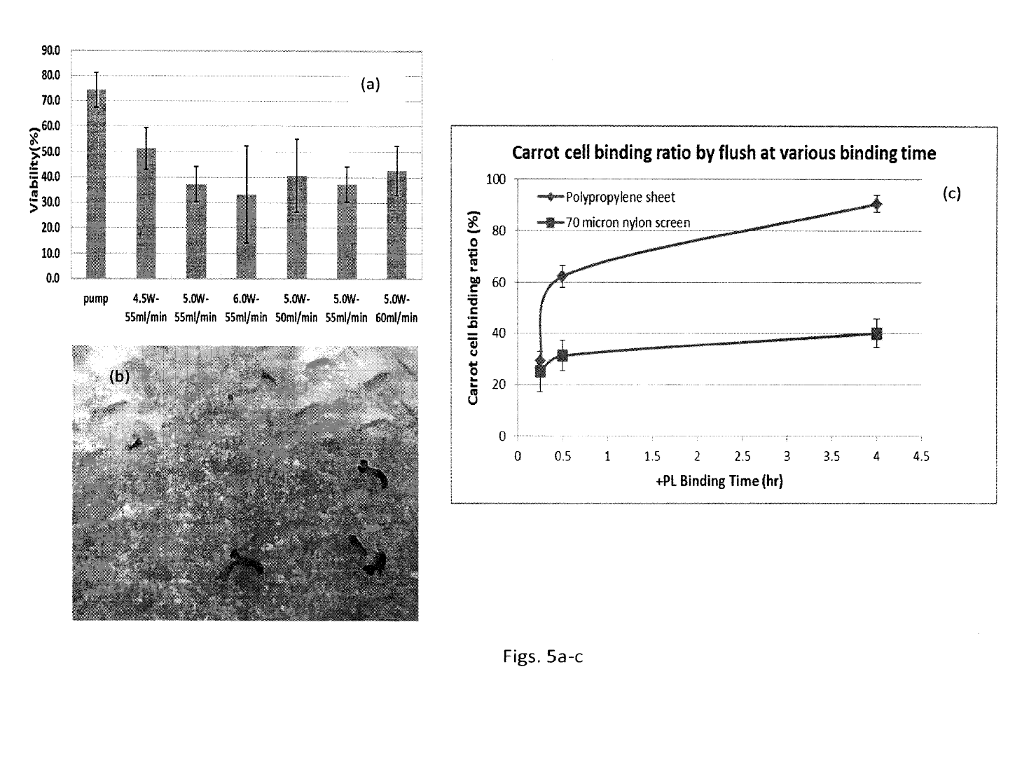

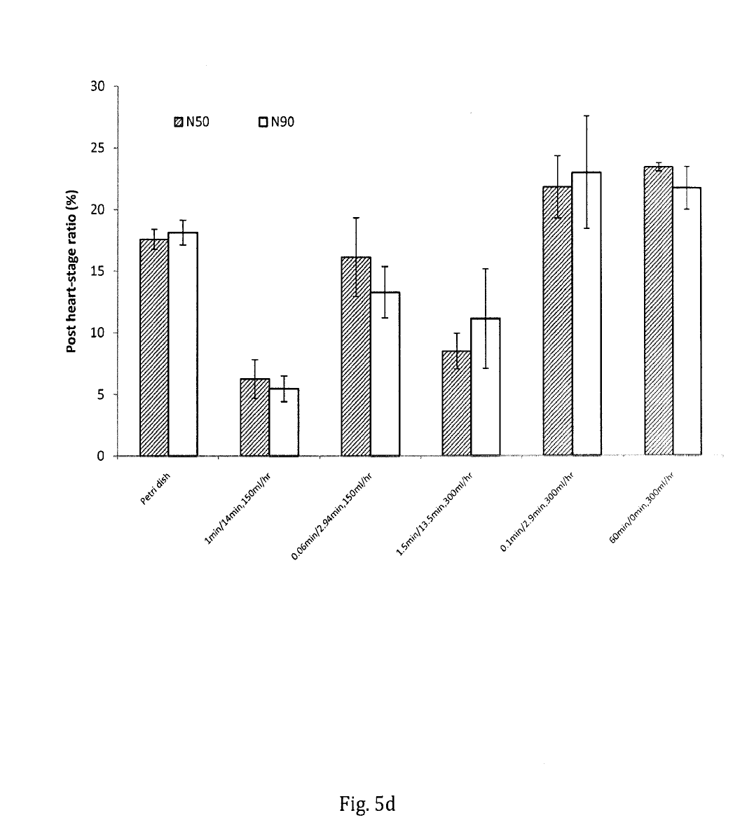

In one example of the methods described herein, undifferentiated carrot cells (Daucus sp.) were suspended in their culture medium and sprayed aseptically into a mist bioreactor to demonstrate they can both survive the spray inoculation and also develop into embryos. Cell viability was determined through use of two different viability stains, fluorescein diacetate and phenosafranin. FIG. 5a shows viability results after inoculation through the ultrasonic misting head of the reactor and FIG. 5b shows a developed embryo. Cells were inoculated onto PL coated strips 10 of either nylon mesh or polypropylene sheeting and incubated up to four hours at room temperature. To measure if cells were indeed attached, the sheet was either rinsed/flushed with 10 flushes of 0.3 mL (total=3 mL) of water in an attempt to dislodge the cells. Those remaining after this wash were deemed to be attached; results are shown in FIG. 5c.

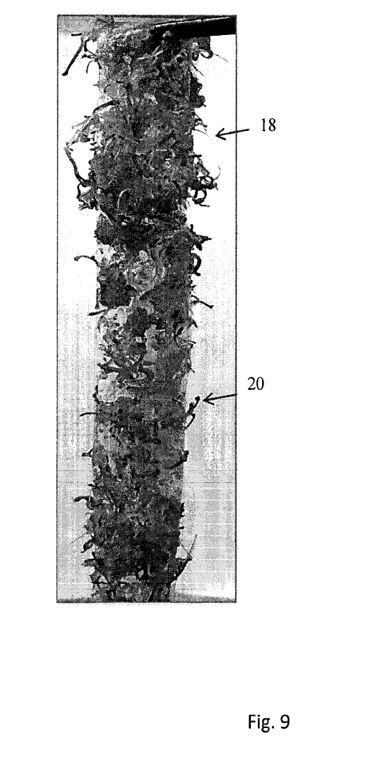

The mist bioreactor as described herein is mainly composed of a growth chamber 12 made of disposable plastic, an ultrasonic nozzle with a conical tip, gas inlet, medium reservoir, timer and peristaltic pump. In the example above, the carrot cells were sprayed through the ultrasonic nozzle onto nylon screens placed in the growth chamber, fed with B5 medium mist (although any plant culture medium can be used as appropriate for different plant species) in different feeding cycles and harvested after two weeks to measure both cell viability and development into embryos. After inoculation using 4.5 watts of ultrasonic power, 51.2% cells remained viable (FIG. 5a). In FIG. 5a, “55 ML/MIN” refers to the volumetric nutrient liquid culture medium feed rate as it enters the input of the bioreactor through the misting nozzle and then sprayed into the actual culture vessel (the plastic bag). Using a feeding cycle of 1 min mist on every 15 min at 38 ml/min, the total embryos produced per gram fresh weight were 11,322 and 10,138 for nylon screens with openings of 50 micron (CMN50) and 90 micron (CMN90), respectively. Of these embryos, there was no difference in which nylon screen was used; about 3.5% were developed to the torpedo or cotyledon stage. When a more frequent misting cycle was used that fed the same volume of medium per hour, embryo formation improved with increased nutrient flow rate and increased misting cycle (FIG. 5d). For example, frequent misting at 0.1 min/2.9 min at 300 mL/hr was better than 1.5 min/13.5 min at 300 mL/hr, and 300 mL/hr was better than 150 mL/hr. However, when continuous misting (60 min/0 min, 300 mL/hr) was used, there was no further improvement in embryo development, although it was also not detrimental to embryo development (FIG. 5d). On the other hand, the yield of embryos beyond the heart stage nearly quadrupled and was slightly greater than standard embryogenesis in Petri dishes. It was found that cells attached to a strip 10 and then hung inside a mist bioreactor will develop into embryos and then small plantlets with roots 18 and shoots 20 after about 21 days (FIG. 9).

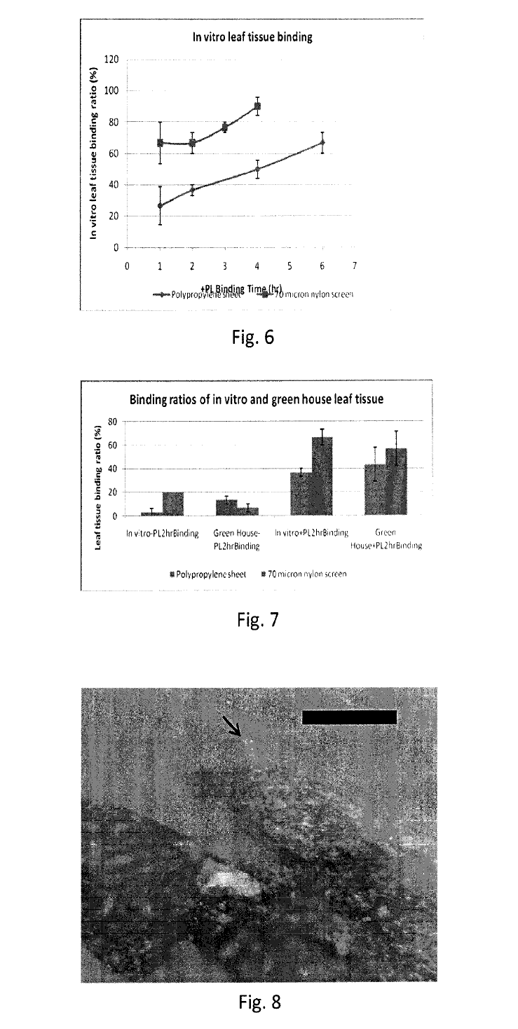

Micro-Propagation of Plant Leafy Tissue In another example of the methods described herein, small (−0.25 cm²) sections of leaves from in vitro cultured Artemisia annua plants were placed manually onto strips 10 of either PL coated polypropylene sheeting or 70 μm nylon mesh and incubated in culture medium B5 up to six hours at room temperature. The best adhesion occurred in sucrose without any medium salts. Adhered tissues were then subjected to flushing with water using a pipette. After 10 flushes of 0.3 mL (total=3 mL) of media were washed over the tissues, those that remained attached were counted. Leafy tissues stuck to PL coated nylon and polypropylene and the % attachment increased as incubation time increased (FIG. 6). Although it appears that nylon was the better attachment material that may be due to the fact that it is a mesh. Nevertheless, either type of material, in either form will work. Greenhouse-grown leaves (GH leaves), which have a more developed waxy cuticle and may not be as amenable to attachment, were nevertheless tested for their ability to attach. Surprisingly, GH leafy tissue also bound well and was similar in attachment levels to that of the in vitro-grown leaves. FIG. 7 shows results after a two hour incubation on the PL coated substrate. The mesh coated (+PL) and uncoated (−PL) showed better attachment than the sheeting; sheeting may however be preferred for rooted plantlets as roots will not be damaged upon harvest as they might be using mesh. On the other hand, smaller opening mesh that prevents roots from growing through mesh openings less than the diameter of the plant roots or hairs could also be used to obviate this concern.

The surface of leaves have protrusions called trichomes. In A. annua there are two main types: filamentous and glandular. When viewed under a light microscope, it appeared that the binding tether was by the filamentous trichomes, as well as by other as yet unidentified nontrichome mechanisms (which may be charge related). FIG. 8 shows a filamentous trichome attached to a PL-coated sheet. Attachment was verified by tapping the microscope slide and observing lateral movement of the green tissue off the attachment point (arrow in FIG. 8). Smaller portions of leaves that do not contain trichomes also attach to coated strips and mesh.

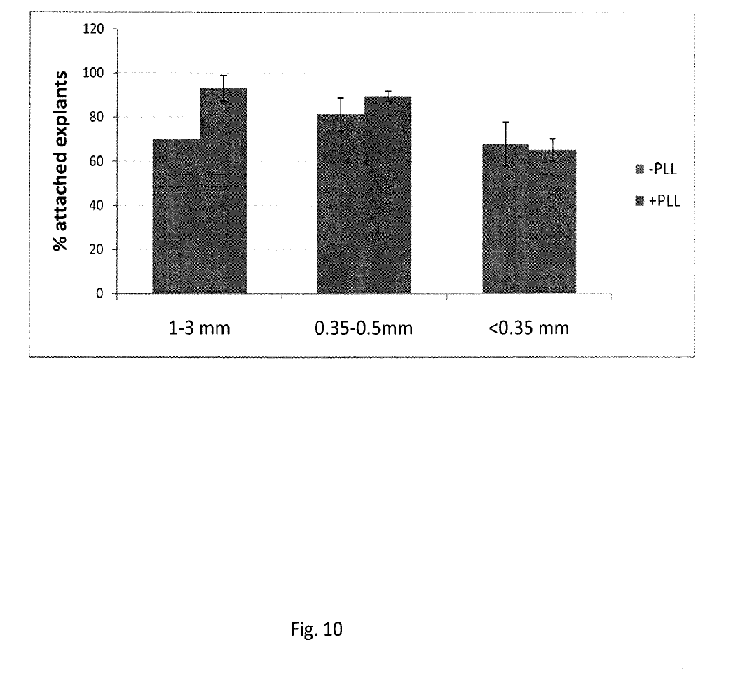

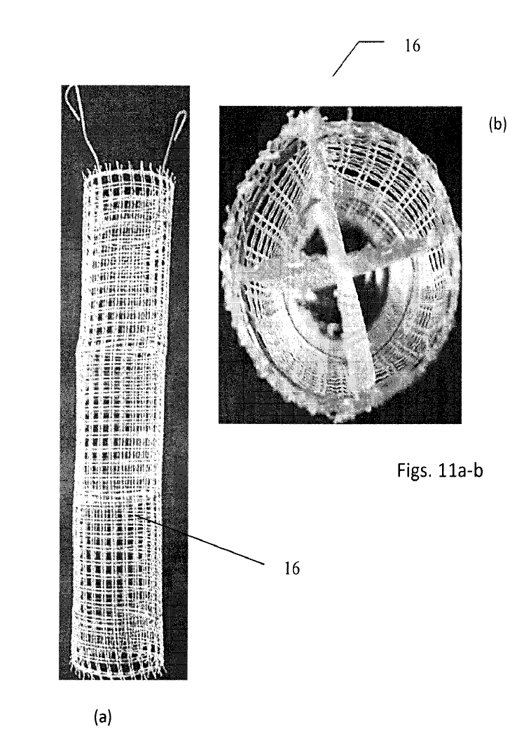

In another example, A. annua leaves were pulsed in a blender and sieved to 3 different sizes. Each group of explants was then manually pipetted onto a strip 10 of N70 mesh±PLL, weighed, and then hung inside the mist bioreactor and misted with culture medium for 24 hr (300 mL/hr during 18 sec mist on and 2 min 42 sec mist off). Strips 10 were then removed and explant weight remaining was measured to determine amount lost during misting. Explants remaining on the strip were deemed to be attached and data are shown as % attached in FIG. 10.

### Culturing of Filamentous Fungi:

In another embodiment of the methods described herein, a relatively open mesh 3-D trellis 16 coated with PL is schematically shown in FIGS. 11a-b. The trellis 16 comprises, for example, polypropylene, nylon, polycarbonate, or polystyrene, and provides attachment points and subsequent internal support for the resulting fungal biomass increase that would ensue. Filamentous fungi are similar in morphology to roots and there is extensive research that shows that a fine mist is superb at penetrating deep filamentous beds whereby excellent mass transport of both gases and nutrients are possible (see, for example, Towler, M. J. et al. (2007), “Using an aerosol deposition model to increase hairy root growth in a mist reactor, “Biotechnology & Bioengineering, 96:881-891; Weathers et al. 2008, Weathers P. J. et al. (2008), “Mist reactors: principles, comparison of various systems, case studies,” Electronic Journal of Integrated Biology 3:29-37; Wyslouzil, B. E., et al., “Mist Deposition onto Hairy Root Cultures: Aerosol Modeling and Experiments,” Biotechnol. Prog. 13:185-194, each incorporated by reference herein). Inoculation of the reactor can be aseptically achieved using the same methods shown effective for plant cells as described above or by immersion of the trellis 16 in a suspension of spores or cells with sufficient time allowed for attachment to occur. Medium is then drained and aeroponic (misting) culture can begin. Similar to other SSFs, culture media would drip off of the biomass attached and growing on the PL trellis 16 and accumulate in the sump of the mist bioreactor for recycle into misting feed. Desired product produced by fungi, such as small molecules including antibiotics, accumulates in the culture medium in the reactor sump. Periodically, it can be extracted from the medium to obviate any feedback inhibition.

The advantages of the methods of these teachings include: 1) The viable spray inoculation of cells into the reactor chamber enables axenic culture of the desired species within the already sterilized mist reactor; 2) Attachment of cells and plant organ bits to the positively-charged polymer-coated plastic or nylon support structures enables localization of them for organized eventual harvest. Previously, hairy roots were attached to a coated trellis for purposes of amassing a lot of root biomass within a reactor that was being fed aeroponically (that is, by mist). No development to intact plants was considered, unit plantlets were to be developed or harvested, and indeed that end result would not work in that embodiment because the biomass would have become so entangled that a healthy harvest of intact acclimatized plantlets would have been impossible; 3) Although cells and tissues may eventually stick to surfaces lacking the positively-charged polymer coating, the presence of the positively-charged polymer coating greatly speeds and enhances the attachment (see FIGS. 5c and 7) and makes unlikely the dislodging of cells or tissues from any liquid sheer stress within a reactor. In addition, the positively-charged polymer-coated support structures are disposable; 4) The vertical culture of plantlets within the mist reactor (FIG. 2) on the support structures, minimizes square foot costs, thereby reducing production costs. This enables not only the production of fully intact plants, but also provides an easy and simple harvesting system off linear support structures using either manual labor or by a machine. The reactor production system is also inexpensive. Furthermore, any plant morphology can be accommodated, and by changing reactor conditions (nutrients, phytohormones, light, relative humidity, CO₂ etc.) starting plantlets can be completely and easily acclimatized to plants ready for the field. Fully acclimatized plants can be produced from either cell or tissue inoculum. This is all readily done using the mist reactor as currently designed for larger scale; and 5) The mist reactor described herein improves solid state fermentation for culturing fungi by enabling more stringent axenic culture for production of products requiring more stringent conditions due to regulatory concerns, e.g. for human health.

Micropropagation of both plants and fungi can now be effectively scaled-up using methods disclosed herein. This adherent-plant-on-structural support technology could also be used to improve other scalable micropropagation “reactors” currently in use, e.g. horizontal rocker boxes. The technology allows for an organized culture of the tissues versus the currently used bulk mass that grows up within each culture box.

Although the invention has been described with respect to various embodiments, it should be realized that this invention is also capable of a wide variety of further and other embodiments within the spirit and the scope of the appended claims.

What is claimed is:

1. A method of micropropagating plants, the method comprising:
coating at least part of a support structure with a positively-charged polymer;
attaching plant materials to said polymer-coated support structure;
placing one or more of said polymer-coated support structure inside of a growth chamber of a plant bioreactor;
allowing said plant materials to develop into plantlets;
optionally acclimating said plantlets to an environment outside of said plant bioreactor; and
harvesting said plantlets.

2. The method of claim 1, wherein said support structure comprises a flexible sheeting, film or mesh.

3. The method of claim 2, wherein said support structure is in the form of a strip, sheet or tube.

4. The method of claim 1, wherein said positively charged polymer comprises poly-L-lysine, poly-Larginine, poly-L-omithine or poly-L-histidine.

2. The method of claim 1, wherein said plant materials are plant cells.

3. The method of claim 1, wherein said plant materials are sections of plant leaves, stems or reproductive material.

4. The method of claim 1, wherein said support structure comprises polypropylene, polycarbonate, nylon, or polystyrene.

5. The method of claim 1, further comprising the step of, prior to coating said at least one side of said support structure with said positively-charged polymer, sterilizing said support structure.

6. The method of claim 1, wherein the step of attaching said plant materials to said support structure comprises immersion of said support structure in a solution of said plant materials and waiting for a period of time for attachment to occur.

7. The method of claim 6, wherein said period of time is at least 30 minutes.

8. The method of claim 6, wherein said immersion of said support structure in said solution of said plant materials comprises spraying said solution of said plant materials onto said support structure.

9. The method of claim 8, wherein said spraying is through an ultrasonic nozzle.

10. The method of claim 1, wherein the step of attaching said plant materials to said support structure comprises manually placing or pipetting said plant materials on said support structure.

11. The method of claim 1, wherein said growth chamber comprises disposable plastic.

12. The method of claim 1, wherein the step of coating said at least part of said support structure comprises coating at least one side of said support structure substantially in its entirety.

13. The method of claim 1, wherein the step of coating said at least part of said support structure comprises coating at least one side of said support structure in organized segments or patterns.

14. The method of claim 13, wherein said segments or patterns comprise blocks, triangles, ovals, dots, stripes or zig-zags.

15. A method of cultivating fungi to produce a product, the method comprising:
coating at least part of a support structure with a positively-charged polymer;
attaching fungal materials to said polymer-coated support structure;
placing said support structure inside of a growth chamber of a plant bioreactor;
flowing a culture medium over said attached fungal materials;
optionally allowing said fungal materials to develop into a filamentous form; and
collecting an excreted product of said fungi.

16. The method of claim 15, wherein said fungal materials are fungus cells.

17. The method of claim 15, wherein said fungal materials are fungus spores.

18. The method of claim 15, wherein said support structure comprises polypropylene, nylon, polycarbonate, or polystyrene.

19. The method of claim 15, wherein said support structure is in the form of a trellis.

20. The method of claim 15, wherein the step of attaching said fungal materials to said support structure comprises immersion of said support structure in a solution of said fungal materials and waiting for a period of time for attachment to occur.

21. The method of claim 20, wherein the step of immersion of said support structure in said solution of fungal materials comprises spraying said fungal materials onto said support structure with a solution of said fungal materials.

22. The method of claim 21, wherein said spraying is through an ultrasonic nozzle.

23. The method of claim 15, wherein said growth chamber comprises disposable plastic.

24. A plantlet as made by the method of claim 1.

25. An excreted product as made by the method of claim 15.
